(19) United States
(12) Patent Application Publication
(10) Pub. No.: US 2026/0108390 A1
(43) Pub. Date: Apr. 23, 2026

(54) GANTRY FOR THE DELIVERY ARM OF A LASER HEAD

(71) Applicant: ALCON INC., Fribourg (CH)

(72) Inventors: Geoffrey C. JAWIDZIK, Mission Viejo, CA (US); Brandon L. SPOELSTRA, Trabuco Canyon, CA (US); Scott NEWTON, Costa Mesa, CA (US)

(21) Appl. No.: 19/351,408

(22) Filed: Oct. 7, 2025

Related U.S. Application Data
(60) Provisional application No. 63/708,364, filed on Oct. 17, 2024.

(51) Int. Cl. A61F 9/008; A61B 34/00; B25J 13/06; B25J 13/08; B25J 15/00; B25J 19/00

(52) U.S. Cl. CPC A61F 9/008; A61B 34/74; B25J 13/065; B25J 13/088; B25J 15/0019; B25J 19/0004; A61B 2034/742

(57) ABSTRACT

In one or more embodiments, an ophthalmic system includes a chassis, a delivery arm, a gantry, and a computer. The chassis is coupled to a laser source that can generate a laser beam. The delivery arm is coupled to a laser head that can direct the laser beam towards a target. The gantry includes a positioning system and an adjustment system. The positioning system facilitates motion of the delivery arm and includes an angular system that can facilitate angular motion of the delivery arm. The adjustment system includes linear subsystems that can facilitate motion of the delivery arm. The computer system detects a request for movement of the delivery arm and determines an angle of angular movement of the delivery arm relative to the chassis. The computer system generates an instruction for the requested movement according to the request and the angle and sends the instruction to the adjustment system.

### FIELD

Embodiments of the present disclosure relate to laser systems with a gantry for the delivery arm of a laser head.

### BACKGROUND

Laser medical systems typically include a delivery arm with a laser head that directs a laser beam to the treatment site of the patient. The laser head is placed at a specific position relative to the treatment site in order to treat the patient. The delivery arm may be adjusted to place the laser head at the proper position.

### SUMMARY

In one or more embodiments, an ophthalmic system includes a chassis, a delivery arm, a gantry, and a computer. The chassis is coupled to a laser source that can generate a laser beam. The delivery arm is coupled to a laser head that is optically coupled to the laser source. The laser head can direct the laser beam towards a target. The gantry is coupled to the delivery arm and the chassis. The gantry includes a positioning system and an adjustment system. The positioning system facilitates motion of the delivery arm and includes an angular system that can facilitate angular motion of the delivery arm. The adjustment system facilitates motion of the delivery arm and includes linear subsystems. Each linear subsystem can facilitate motion of the delivery arm. The computer system detects a request for a requested movement of the delivery arm and determines an angle of an angular movement of the delivery arm relative to the chassis. The computer system generates an instruction for the requested movement according to the request and the angle of the angular movement of the delivery arm and sends the instruction for the requested movement to the adjustment system.

One or more embodiments may include none, one, some, or all of the following features:
The computer system may generate the instruction for the requested movement by: translating, using the angle, the requested movement expressed in a delivery arm coordinate system to a translated requested movement expressed in a chassis coordinate system; and generating the instruction for the requested movement using the translated requested movement expressed in the chassis coordinate system.
The ophthalmic system has a chassis coordinate system and a delivery arm coordinate system. The chassis coordinate system relative to the chassis has a first axis and a second axis, where the second axis is at a first angle to the first axis. The delivery arm coordinate system relative to the delivery arm has a fourth axis and a fifth axis, where the fifth axis is at a second angle to the fourth axis. The requested movement is expressed in the delivery arm coordinate system and includes a fourth movement relative to the fourth axis and a fifth movement relative to the fifth axis. The computer system may generate the instruction for the requested movement by: calculating a first movement relative to the first axis according to the fourth movement, the fifth movement, and the angle of the angular movement of the delivery arm; calculating a second movement relative to the second axis according to the fourth movement, the fifth movement, and the angle of the angular movement of the delivery arm; and generating the instruction for the requested movement according to the first movement and the second movement.
The linear subsystems may include a first subsystem and a second subsystem. The first subsystem moves the delivery arm in a first direction relative to a first axis of a chassis coordinate system relative to the chassis. The second subsystem moves the delivery arm in a second direction relative to a second axis of the chassis coordinate system.
The linear subsystems may include a third subsystem. The third subsystem moves the delivery arm in a third direction parallel to a third axis, which is defined according to an optical axis of the laser head of the delivery arm.
A linear subsystem may include a stage base and a stage that moves relative to the stage base to facilitate the motion of the delivery arm. The linear subsystem may include an encoder that measures the linear movement of the stage relative to the stage base.
The angular system may include a rotary stage and a rotor that rotates relative to the rotary stage to enable the angular motion of the delivery arm.
The positioning system may include an encoder that measures the angle of the angular movement of the delivery arm.
The angular system may be coupled to a linear subsystem.
The positioning system may include a fourth subsystem that enables a fourth linear motion of the delivery arm in a fourth direction relative to a rotational axis of the delivery arm.
The positioning system may include an angular brake that restricts the angular motion of the delivery arm.
The computer system may detect a request to allow a movement of the delivery arm between a stowed position and a deployed position and may disengage a stowed-state latch to allow the movement of the delivery arm from the stowed position towards the deployed position.
The ophthalmic system may include a latch that restricts movement of the delivery arm from a stowed position.
The ophthalmic system may include a latch that restricts movement of the delivery arm from a deployed position.
The computer system may detect a request to allow the angular motion of the delivery arm and may disengage one or more brakes to allow the angular motion of the delivery arm.

### BRIEF DESCRIPTION OF THE DRAWINGS

FIG. 1 illustrates an example of an ophthalmic system, according to at least one embodiment described in the present disclosure;

FIG. 2 illustrates an example of an ophthalmic system, according to at least one embodiment described in the present disclosure;

FIGS. 3A and 3B illustrate an example of a delivery arm of an ophthalmic system, according to at least one embodiment described in the present disclosure;

FIGS. 4A and 4B illustrate examples of computing systems of an ophthalmic system, according to at least one embodiment described in the present disclosure;

FIG. 5 illustrates an example of a gantry that facilitates movement of a delivery arm, according to at least one embodiment described in the present disclosure;

FIGS. 6 to 7B illustrate an example of a z-subsystem, according to at least one embodiment described in the present disclosure, where FIG. 6 illustrates an example of a z-stage base, and FIGS. 7A and 7B illustrate an example of a z-stage;

FIGS. 8 to 9B illustrate an example of a y-subsystem, according to at least one embodiment described in the present disclosure, where FIG. 8 illustrates an example of a y-stage base, and FIGS. 9A and 9B illustrate an example of a y-stage/x-stage base;

FIGS. 10A to 10D (in combination with FIGS. 9A and 9B) illustrate an example of an x-subsystem and a theta-subsystem, according to at least one embodiment described in the present disclosure, where FIGS. 10A and 10B illustrate an example of an x-stage and a theta-stage, FIGS. 10C and 10D illustrate an example of a theta-stage brake, and FIG. 10E illustrates an example of an angle encoder;

FIGS. 11A to 12D illustrate another example of an x-subsystem, according to at least one embodiment described in the present disclosure, where FIGS. 11A and 11B illustrate an example of a y-stage/x-stage base, and FIGS. 12A and 12B illustrate an example of an x-stage;

FIGS. 13 to 14B illustrate an example of an R-subsystem, according to at least one embodiment described in the present disclosure, where FIG. 13 illustrates an example of an R-stage base, and FIGS. 14A and 14B illustrate an example of an R-stage; and

FIG. 15 illustrates an example of a method for moving a delivery arm of an ophthalmic system, according to at least one embodiment described in the present disclosure.

### DESCRIPTION OF EXAMPLE EMBODIMENTS

Referring now to the description and drawings, one or more example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

The present disclosure relates to an ophthalmic system that includes a gantry for the delivery arm of a laser head. The gantry facilitates movement of the delivery arm to place the laser head at the proper position relative to the eye of the patient. In certain embodiments, the delivery arm may be initially coarsely moved to place the laser head proximate to the patient. The gantry may then be used to make fine adjustments to precisely position the laser head relative to the eye. The initial coarse adjustment may change the position of the delivery arm relative to the ophthalmic system. The gantry may take into account the change in position to effectively make the fine adjustments.

Certain embodiments of the present disclosure may provide improvements over previous iterations of positioning a laser head. For example, the ophthalmic system accounts for changes in the position of the delivery arm relative to the ophthalmic system, which allows for the initial coarse adjustment of the delivery arm. As another example, the gantry may include subsystems that can linearly move and/or rotationally move the delivery arm. As yet another example, the gantry may include one or more latches and/or brakes that restrict (e.g., slow down and/or stop) the movement of the delivery arm. Additionally, certain embodiments of the present disclosure may provide an intuitive interface via which a surgical device may be moved and/or adjusted, even if positioned at different directions or angles relative to a patient during different procedures even within the same surgical setting.

For example, for a given surgical setting, a patient may be wheeled towards the left side of the ophthalmic system at a first acute angle. A button may be depressed on a handle located on or near the laser head of the system, and the laser head may be pulled out using coarse positioning to place the laser head generally over the eye of the patient. A nurse or physician may then use a joystick to fine-tune the position of the laser head and/or dock the laser head to the eye of the patient. After the procedure, the laser head may be returned to a stowed position. Within the same surgical setting, another patient may be wheeled directly in front of the ophthalmic system, and the laser head may be pulled out in a different direction, but still so as to be over the eye of the patient. In the same surgical setting, another patient may be wheeled towards the right side of the ophthalmic system at a wide angle. The button may be depressed on the handle and the laser head may be pulled out for coarse positioning of the laser head over the eye of the patient. A different joystick on the other side of the laser head may be used by the nurse of physician to fine-tune the position of the laser head and dock the laser head to the eye of the patient. After the procedure, the laser head may be returned to the stowed position. In this manner, a surgeon may be provided with a significant amount of flexibility in positioning patients relative to the laser head.

However, in such a circumstance, the relative motion between different gantry devices is complex as only linear actuators are typically used. If the patient is always at the same position, the consistent linear motion works well. The present disclosure provides for complex motion when a joystick or other input device is used to move the laser head, even if the patient is at various angles and the laser head is to move in a non-linear direction.

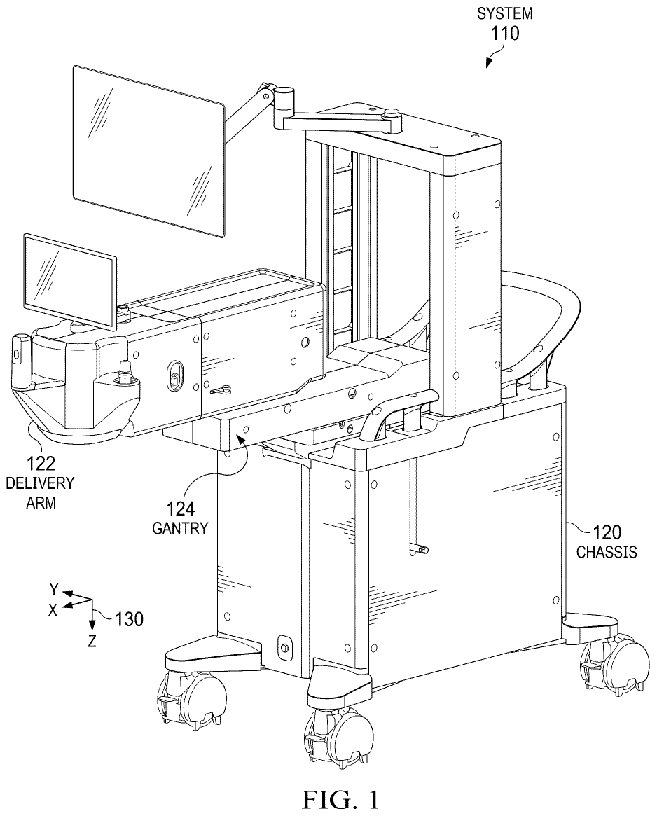

FIG. 1 illustrates an example of an ophthalmic system 110, according to at least one embodiment described in the present disclosure. In the example, the ophthalmic system 110 includes a chassis 120, a delivery arm 122, and a gantry 124, which may be coupled as shown.

As an overview of the example, the chassis 120 includes a laser source that generates a laser beam, and the delivery arm 122 has a laser head that directs the laser beam towards the eye of a patient. The gantry 124 moves the delivery arm 122 such that the laser head is properly located relative to the eye. The gantry 124 facilitates motion (e.g., linear and/or angular motion) of the delivery arm 122 for coarse adjustment and/or for fine adjustment of the delivery arm 222. The coarse adjustment and/or fine adjustment may be performed manually and/or may be automated using an input device such as a joystick. In certain embodiments, the initial coarse positioning is performed manually by manipulating a handle on the delivery arm, and the subsequent fine adjustment is automated using, e.g., a joystick.

For ease of explanation, a chassis coordinate system 130 defined relative to the chassis 120 is shown. The chassis coordinate system 130 has an x-axis, a y-axis, and/or a z-axis, which may have any suitable relative angles among them. For example, the x-axis, the y-axis, and/or the z-axis may be orthogonal to each other. The z-axis may be aligned with or parallel to an optical axis of the ophthalmic system 110, and the x-axis and the y-axis may be orthogonal to the z-axis. As another example, an x-axis, a y-axis, and/or a z-axis may have other suitable angles (e.g., angles in the range of 1 to 179 degrees) relative to each other and may have any suitable geometric relationship with the ophthalmic system 110. Position in the chassis coordinate system 130 may be expressed using an x-coordinate, a y-coordinate, and/or a z-coordinate. Movement in an axial direction may be movement in a direction parallel to an axis. For example, movement in an x-direction may be movement in a direction parallel to the x-axis. Similarly, movement in a y-direction may be movement in a direction parallel to the y-axis, and movement in a z-direction may be movement in a direction parallel to the z-axis.

The ophthalmic system 110 may be any suitable system that may be used to perform a procedure (e.g., a surgical and/or diagnostic procedure) on an eye. Examples of ophthalmic diagnostic procedures include corneal topography, corneal keratometry, intraocular pressure measurement, tear film assessment, and/or eye tissue imaging procedures. Examples of ophthalmic surgical procedures include cataract, refractive, vitreoretinal, and/or other ophthalmic procedures. In certain embodiments, the ophthalmic system 110 may be a cataract system that performs a cataract procedure.

Turning to the components of the ophthalmic system 110, the chassis 120 may support and/or house one or more components of the ophthalmic system 110, e.g., a laser source (not shown), the gantry 124, and/or the delivery arm 122. The delivery arm 122 includes a laser head (not shown) that delivers a laser beam from the laser source. The gantry 124 facilitates movement of the delivery arm 122 in order to position the laser head. The delivery arm 122 may be moved from a stowed position to a deployed position. The stowed position may be, e.g., a retracted position such that the delivery arm 122 is closer to the chassis 120 and/or that all or a portion of the delivery arm 122 is at and/or within the chassis 120. The deployed position may be, e.g., an extended position such that the delivery arm 122 is ready to be aligned with the patient's eye.

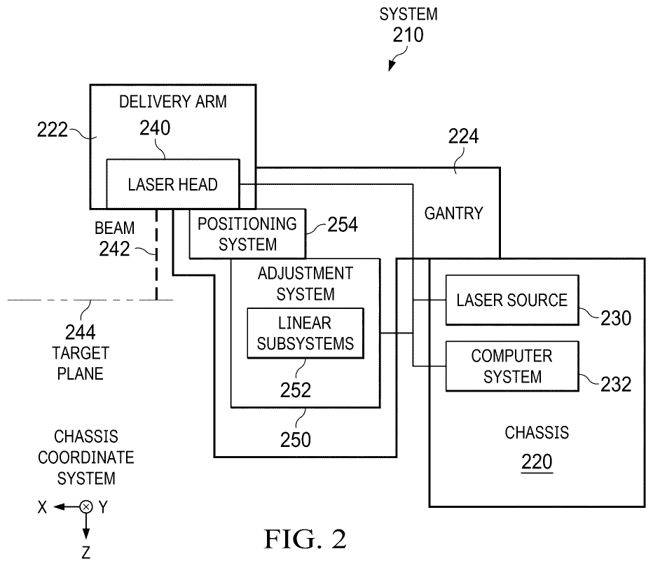

FIG. 2 illustrates an example of an ophthalmic system 210, according to at least one embodiment described in the present disclosure. In the example, the ophthalmic system 210 includes a chassis 220, a delivery arm 222, and a gantry 224, which may be coupled as shown. The chassis 220 includes a laser source 230 and/or a computer system 232, which may be coupled as shown. The delivery arm 222 includes a laser head 240, which may direct a laser beam 242 to a target plane 244. The gantry 224 includes an adjustment system 250 (which includes one or more linear subsystems 252) and/or a positioning system 254, which may be coupled as shown.

As an example of an operation of the ophthalmic system 210, the gantry 224 operates to move the delivery arm 222 such that the eye of the patient is properly located relative to the target plane 244. To move the delivery arm 222, the positioning system 254 of the gantry 224 may facilitate coarse positioning of the delivery arm 222, e.g., angular motion of the delivery arm 222 by a theta angle. After the delivery arm 22 has been coarsely positioned, the adjustment system 250 may facilitate fine adjustment of the delivery arm 222. For example, each linear subsystem 252 of the adjustment system 250 may linearly move the delivery arm 222 in an axial direction, e.g., in response to manipulation of a joystick.

In the example of operation, the computer system 232 allows a user to perform the fine adjustment of the delivery arm 222 using the same motion relative to the delivery arm 222 regardless of the theta angle of the delivery arm 222. For example, a joystick may be moved in the direction of the intended movement whether the delivery arm 222 is at a first theta angle or at a second theta angle that is different from the first theta angle, as described in more detail herein. After the delivery arm 222 has been finely adjusted, the laser source 230 of the chassis 220 generates the laser beam 242, and the laser head 240 of the delivery arm 222 directs the laser beam 242 towards the target plane 244.

Turning to the components of the ophthalmic system 210, the chassis 220 supports and/or houses any suitable components of the ophthalmic system 210. In the illustrated example, the chassis 220 supports and/or houses the laser source 230, the computer system 232, and/or the gantry 224. However, in other examples, the laser source 230, the computer system 232, and/or the gantry 224 may be located elsewhere and/or other components (e.g., the adjustment system 250 and/or the positioning system 254) may be supported and/or housed by the chassis 220. The chassis 220 and/or gantry 224 may support the delivery arm 222 at any suitable height, e.g., with the patient-interfacing component elevated in the range of 30 to 45 inches from the floor.

In certain embodiments, the laser source 230 generates a laser beam. Examples of the laser source 230 include excimer and femtosecond lasers. The laser beam may have any suitable pulse duration, such as in the order of nanoseconds, picoseconds, femtoseconds, or attoseconds. The laser beam may have any suitable wavelength, such as in the range of 150 nanometers (nm) to 20 micrometers (μm). Examples of ranges include the ultraviolet (e.g., in the range of 180 to 400 nm, such as 190 to 195 nm or 345 to 355 nm), visible, or infrared wavelength (e.g., in the range of 1050 to 1250 or 1250 to 1500 nm). The laser beam may process material in any suitable manner, e.g., ablate, incise, or photo-disrupt the material. The laser head 240 of the delivery arm 222 directs the laser beam 242 towards the target plane 244.

The gantry 224 operates to move the delivery arm 222 such that the eye of the patient is properly located relative to the target plane 244. The positioning system 254 of the gantry 224 facilitates linear and/or angular motion of the delivery arm 222. This angular motion may be used for coarse adjustment of the delivery arm 222. The adjustment system 250 of the gantry 224 facilitates motion of the delivery arm 222. For example, each linear subsystem 252 can linearly move the delivery arm 222. This linear motion may be used for fine adjustment of the delivery arm 222 with, e.g., a joystick.

The computer system 232 manages the operation of components of the ophthalmic system 210, such as the delivery arm 222, the gantry 224, the laser source 230, the laser head 240, the adjustment system 250, and/or the positioning system 254. In certain embodiments, the computer system 232 allows for moving the delivery arm 222 using the same action regardless of the theta angle of the delivery arm 222. For example, the computer system 232 detects a request for a requested movement of the delivery arm 222, e.g., detects the manipulation of the joystick. The computer system 232 determines the theta angle of the delivery arm 222, generates an instruction for the requested movement according to the request and the theta angle, and sends the instruction to the adjustment system 250.

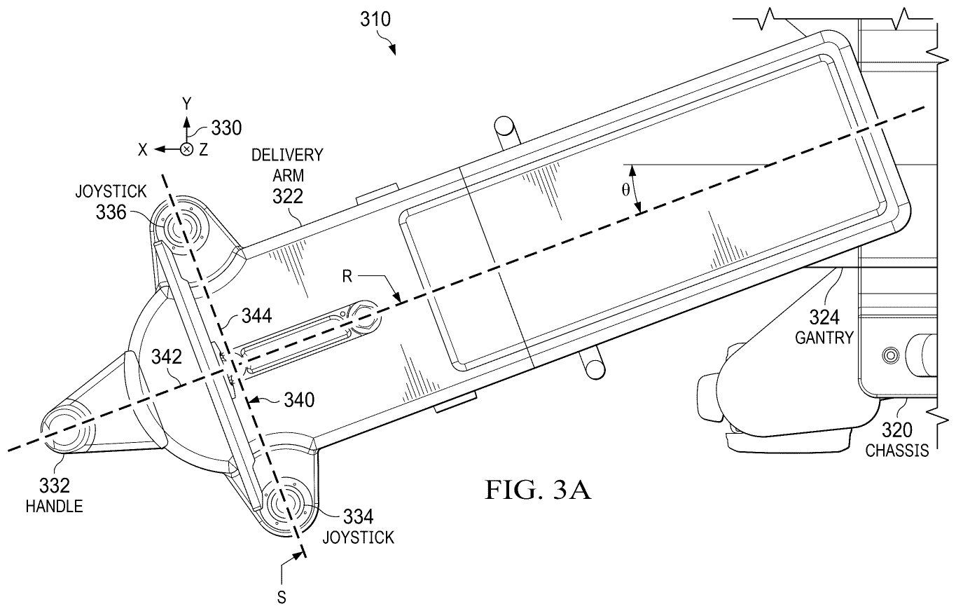

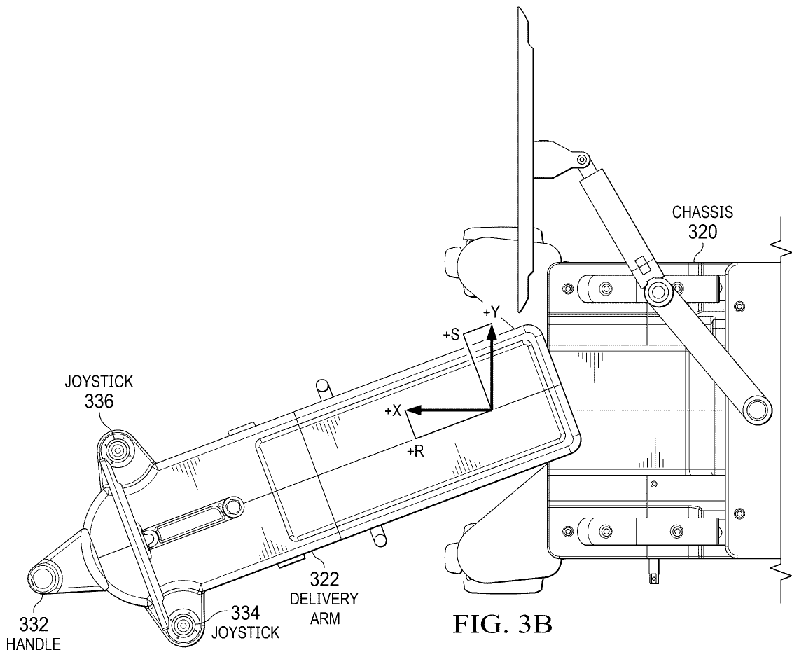

FIGS. 3A and 3B illustrate an example of an ophthalmic system 310, according to at least one embodiment described in the present disclosure. In the example, the ophthalmic system 310 includes a chassis 320, a delivery arm 322, and a gantry 324. The delivery arm 322 includes a handle 332 and one or more joysticks 334 and 336.

For ease of explanation, a chassis coordinate system 330 (examples of which are described herein) is shown with an x-axis, a y-axis, and a z-axis. A delivery arm coordinate system 340 is shown with an R-axis 342 and an S-axis 344 and may have any suitable orientation relative to the delivery arm. In the example, the R-axis 342 is aligned with an axis along the length of the delivery arm 322, and the S-axis 344 is positioned at an angle to the R-axis 342 in an xy-plane of the chassis coordinate system 330. For example, the S-axis 344 may be orthogonal to the R-axis 342 or may be positioned at any other angle (e.g., an angle in the range of 1 to 179 degrees) relative to the R-axis 342. The chassis coordinate system 330 and the delivery arm coordinate system 340 may have any suitable relative orientation. For example, the x-axis may be parallel to the R-axis 342 and the y-axis may be parallel to the S-axis 344 when the delivery arm 322 is in a neutral or stowed position.

As described above, a position of the chassis coordinate system 330 may be expressed using an x-coordinate, a y-coordinate, and/or a z-coordinate, and movement in the x-direction, y-direction, or z-direction may be movement in a direction parallel to the x-axis, the y-axis, or the z-axis, respectively. Similarly, a position of the delivery arm coordinate system 340 may be expressed with an R-coordinate and an S-coordinate. Movement in the R-direction or S-direction may be movement in a direction parallel to the R-axis or the S-axis, respectively.

FIG. 3A may be used to describe coarse adjustment of the delivery arm 322. In certain embodiments, a positioning system facilitates coarse adjustment of the delivery arm 322 by facilitating angular movement of the delivery arm 322 through the theta angle and/or facilitating linear R-movement of the delivery arm 322 in an R-direction. In an example, the handle 332 may be used to manually move the delivery arm 322. In other examples, the handle 332 may be used to actuate automated movement of the delivery arm.

FIG. 3B may be used to describe fine adjustment of the delivery arm 322. In certain embodiments, an adjustment system facilitates fine adjustment of the delivery arm 322. In the embodiments, a user may manipulate a joystick 334 or 336 to send a request for a requested movement. For example, the joystick 334 or 336 may be tilted in a direction to move the delivery arm 322 in that same direction. The user may use the same manipulation of the joystick 334 or 336 relative to the delivery arm 322, regardless of the theta angle of the delivery arm 322. In the example, the manipulation may be made relative to the delivery arm coordinate system 340, such that the requested movement may be described by coordinates (R, S).

In certain embodiments, a computer system generates an instruction for the requested movement by translating, using the theta angle, the requested movement expressed in the delivery arm coordinate system 340 to a requested movement expressed in the chassis coordinate system 330. In the embodiments, the computer system may calculate the x-movement and the y-movement that yields the requested R-movement and S-movement from the R-movement, the S-movement, and the theta angle. For example, the computer system may use the following equations:

$$X = R cos ( theta ) + S sin ( theta )$$

$$Y = - R sin ( theta ) + S cos ( theta )$$

The computer system may then generate the instruction for the requested movement using the x-movement and the y-movement.

For example, a user (e.g. a nurse, technician, or physician) may use the joystick 336 to move the delivery arm 322, and may be used to moving the delivery arm 322 in a manner square with how the user is oriented. However, the motion is converted to a combination of motions from various linear motions from the gantry 324. For example, the user may be sitting square with the delivery arm 322 as shown in FIG. 3A. However, moving the delivery arm 322 in the x-direction or y-direction in such a circumstance would not be direct linear motion relative to the user. Instead, using at least some embodiments of the present disclosure the user is able to move the joystick 336 in a direction that is square with their orientation (e.g., the R direction or S direction), and the ophthalmic system may employ a combination of both x- and y-direction motion by operating linear motion actuators in both directions in a coordinated manner within the gantry 324. Additionally, by providing both joysticks 334 and 336, the user is able to orient themselves (and the patient) in any position or orientation which is advantageous given the procedure, the surgical setting, and/or any other preferences of the user.

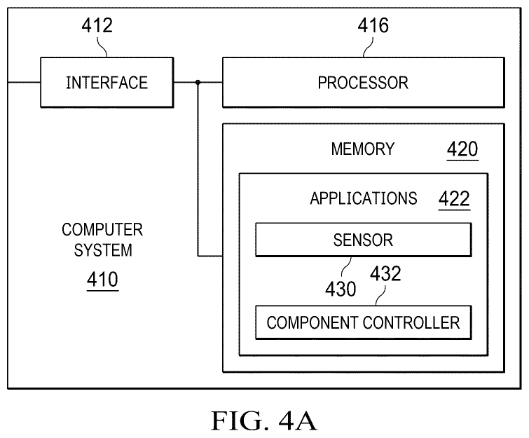

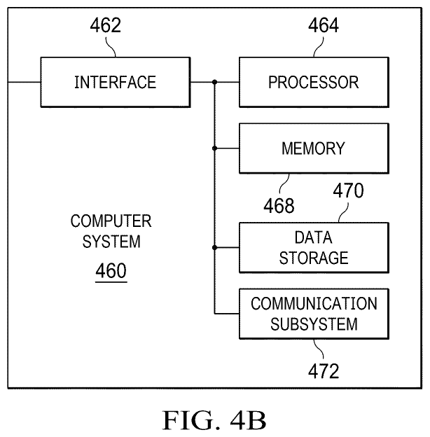

FIGS. 4A and 4B illustrate examples of computing systems 410 and 460 of an ophthalmic system, according to at least one embodiment described in the present disclosure.

FIG. 4A illustrates an example of a computing system 410, according to at least one embodiment described in the present disclosure. The computer system 410 includes an interface 412, a processor 416, and/or a memory 420, any or all of which may be communicatively coupled. The memory 420 stores applications 422, e.g., a sensor application 430 and/or a component controller application 432.

In certain embodiments, the sensor application 430 receives signals from sensors of the ophthalmic system. For example, the sensor application 430 receives a signal from a joystick indicating a direction of movement. As another example, the sensor application 430 receives a signal from an encoder, such as a linear encoder or an angle encoder. The signal from a linear encoder may indicate an amount of linear movement. The signal from an angle encoder may indicate an amount of angular movement, e.g., the amount of a theta angle. The sensor application 430 may provide information from the signals to the component controller application 432. In certain embodiments, the component controller application 432 controls the components of the ophthalmic system.

FIG. 4B illustrates another example of a computing system 460, according to at least one embodiment described in the present disclosure. The computing system 460 may include an interface 462, a processor 464, a memory 468, a data storage 470, and/or a communication subsystem 472, any or all of which may be communicatively coupled. Any or all of the computing system 460 may be implemented as computer hardware, computer software, and/or a combination of computer hardware and computer software. Any or all of the system 100 of FIG. 1 may be implemented as a computing system consistent with the computing system 460.

In the example, the interface 462 may receive input to the computing system 460 and/or send output from the computing system 460, and may be used to exchange information between, e.g., software, hardware, one or more peripheral devices, one or more users, and/or any suitable combinations of any of the preceding. A user interface is a type of interface that a user can utilize to communicate with (e.g., send input to and/or receive output from) the computing system 460. Examples of user interfaces include displays, Graphical User Interfaces (GUIs), touchscreens, foot pedals, keyboards, computer mouses (or mice), joysticks, buttons, gesture sensors, microphones, and speakers.

Generally, the processor 464 may include any suitable special-purpose or general-purpose computer, computing entity, or processing device including various computer hardware or software modules and may be configured to execute instructions stored on any applicable computer-readable storage media. For example, the processor 464 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor, the processor 464 may include any number of processors distributed across any number of network or physical locations that are configured to perform individually or collectively any number of operations described in the present disclosure.

The processor 464 may perform any suitable operations. In some embodiments, the processor 464 may interpret and/or execute program instructions and/or process data stored in the memory 468, the data storage 470, or the memory 468 and the data storage 470. In some embodiments, the processor 464 may fetch program instructions from the data storage 470 and load the program instructions into the memory 468. After the program instructions are loaded into the memory 468, the processor 464 may execute the program instructions, such as instructions to perform any of the methods disclosed herein, respectively.

The memory 468 and the data storage 470 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 464.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 464 to perform a certain operation or group of operations.

The communication subsystem 472 may include any component, device, system, or combination thereof that is configured to transmit, receive, and/or otherwise exchange information over a network in order to communicate with any suitable entity, such as with other devices at other locations or at the same location or even within the same system. The communication subsystem 472 may provide for communication among the devices described in the present disclosure, communication networks, computing devices, and other systems. For example, the communication subsystem 472 may allow the system 460 to communicate with other systems, such as other computing devices and/or networks. In some embodiments, the communication subsystem 472 may include a modem, a network card (wireless or wired), an optical communication device, an infrared communication device, a wireless communication device (such as an antenna), and/or chipset. Examples of communication subsystem 472 include a Bluetooth device, an 802.6 device (e.g., that can communicate with a Metropolitan Area Network (MAN)), a WiFi device, a WiMax device, cellular communication facilities, and/or the like.

One skilled in the art will recognize that modifications, additions, or omissions may be made to the system 460 without departing from the scope of the present disclosure. For example, the system 460 may include more or fewer components than those explicitly illustrated and described.

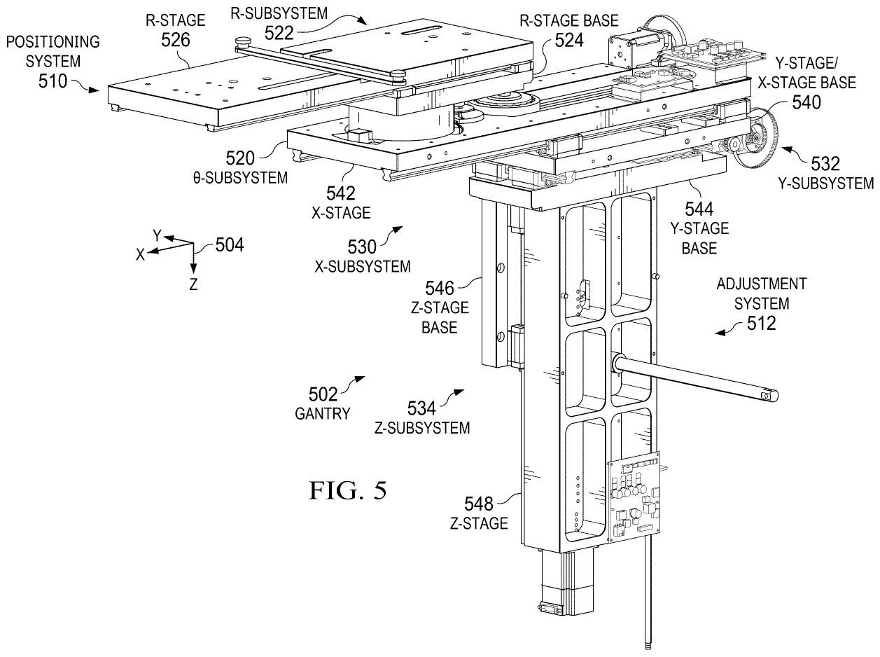

FIG. 5 illustrates an example of a gantry 502 that facilitates movement of a delivery arm (not shown), according to at least one embodiment described in the present disclosure. In the example, the gantry 502 is shown relative to a chassis coordinate system 504, which indicates an x-direction, a y-direction, and a z-direction. The gantry 502 includes a positioning system 510 and an adjustment system 512, which may be coupled as shown. The positioning system 510 includes an angular subsystem, e.g., a theta-subsystem 520, and a linear subsystem, e.g., an R-subsystem 522, which may be coupled as shown. The R-subsystem 522 includes an R-stage base 524 (which may support the theta-subsystem 520) and an R-stage 526, which may be coupled as shown. The adjustment system 512 comprises linear subsystems, e.g., an x-subsystem 530, a y-subsystem 532, and/or a z-subsystem 534, which may be coupled as shown. In the example, the x-subsystem 530 includes a y-stage/x-stage base 540 and an x-stage 542, the y-subsystem 532 includes a y-stage base 544 and the y-stage/x-stage base 540, and the z-subsystem 534 includes a z-stage base 546 and a z-stage 548, which may be coupled as shown.

The positioning system 510 facilitates angular and/or linear motion of the delivery arm and may be used for coarse adjustment of the delivery arm. The theta-subsystem 520 of the positioning system 510 facilitates angular motion. The theta-subsystem 520 may comprise any suitable device, e.g., a rotary stage and a rotor that rotates relative to the rotary stage to facilitate angular motion of the delivery arm, as described in more detail below. The R-subsystem 522 facilitates linear motion of the delivery arm, e.g., motion in an R-direction parallel to an R-axis of the delivery arm, as described in more detail below.

The adjustment system 512 facilitates motion of the delivery arm and may be used for fine adjustment of the delivery arm. Each linear subsystem of the adjustment system 512 can linearly move the delivery arm in, e.g., an axial direction. For example, the x-subsystem 530 can move the delivery arm in the x-direction, the y-subsystem 532 can move the delivery arm in the y-direction, and the z-subsystem 534 can move the delivery arm in the z-direction. A linear subsystem may comprise any suitable device that can linearly move an object. For example, a linear subsystem may comprise a stage base and a stage that can move relative to the stage base to move the delivery arm, as described in more detail below.

A subsystem may include an encoder that measures the movement (e.g., angular and/or linear movement) of the stage relative to the stage base. In certain embodiments, a subsystem may include an encoder that measures the linear movement of the stage relative to the stage base. For example, an encoder may be coupled via a screw or a mechanical gear and may translate rotary movement of the screw or the gear to a linear measurement. As another example, an encoder may include an encoder strip (e.g., a magnetic strip) and an encoder sensor (e.g., a magnetic read-head). The strip may move relative to the sensor, or the sensor may move relative to the strip. The sensor may detect the relative motion to yield a linear measurement of the motion. In certain embodiments, a subsystem may include an encoder that measures the angular movement of the stage relative to the stage base. For example, an encoder may include an annular magnet mounted on a rotating shaft and may include an array of magnetic sensors statically mounted relative to the shaft. The sensors detect and record movement of the shaft relative to the annular magnet to measure angular movement.

A subsystem may include a brake that restricts (e.g., slows down and/or stops) the movement (e.g., angular and/or linear movement) of the stage relative to the stage base. For example, the brake may mechanically stop the stage from moving relative to the stage base. As another example, the brake may apply a voltage to magnets that stop the stage from moving relative to the stage base. As yet another example, a magnet may keep a brake open to allow for movement and may release the brake to restrict movement (e.g., an electromagnet may, when powered, keep the brake open and when no longer powered, release the brake to restrict movement). In certain embodiments, a computer system may control one or more brakes of the system.

By way of example, in some embodiments, a user may utilize the positioning system 510 to move the laser head over the eye of a patient at an angle to the adjustment system 512. After positioning the laser head, the user may use a joystick or other input to move the laser head in a direction that causes both the x-subsystem 530 and the y-subsystem 532 to be engaged in a coordinated manner. For example, if the laser head is positioned at an angle theta and the user were to move the laser head directly in the R direction, the x-subsystem may be actuated by an amount proportional to R cos (theta) and the y-subsystem may be actuated by an amount proportional to −R sin (theta).

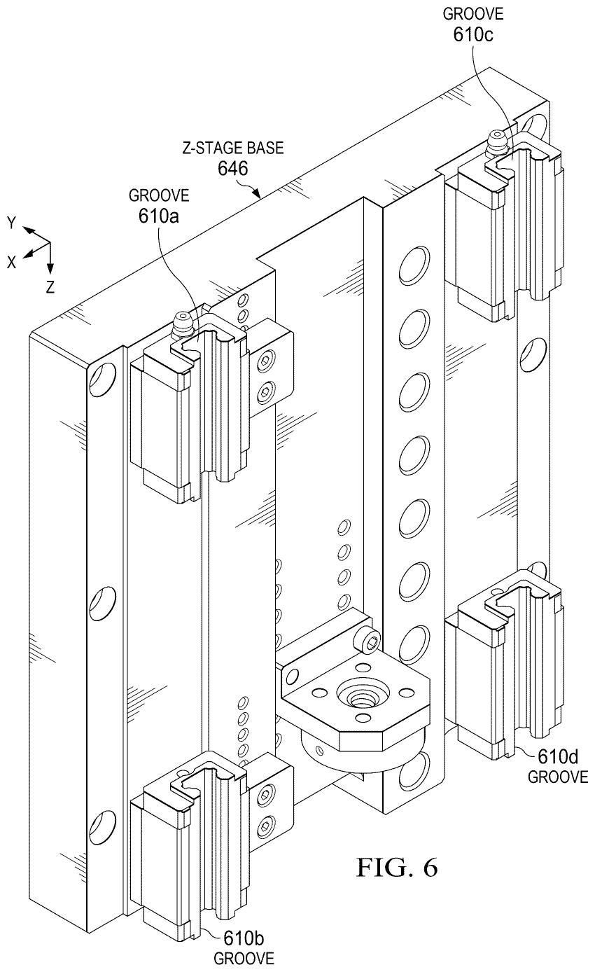

FIGS. 6 to 16B describe examples of motion subsystems that facilitate linear and/or angular motion, according to at least one embodiment described in the present disclosure. A motion subsystem may comprise any suitable components (which may be coupled as shown) arranged to move an object. In the examples, the motion subsystems generally include a stage base and a stage that can move (e.g., linearly and/or rotationally) relative to the stage base. The stage base and the stage may be coupled in any suitable manner. For example, in a linear subsystem, one part (e.g., the stage base or the stage) may have one or more rail(s), and the other part (e.g., the stage or the stage base, respectively) may have one or more groove(s) that are aligned with the rail(s). Each groove may receive a rail and allow the rail to move relative to the groove. While not explicitly described, it will be appreciated that other features may be included to facilitate smooth motion, such as bearings, lubrication, or other features to facilitate motion between a given stage and the associated stage base. A motion subsystem may include other features that affect motion, e.g., a motor that provides motion, a brake or a latch that restricts motion, and/or an encoder that measures motion, as described in more detail below.

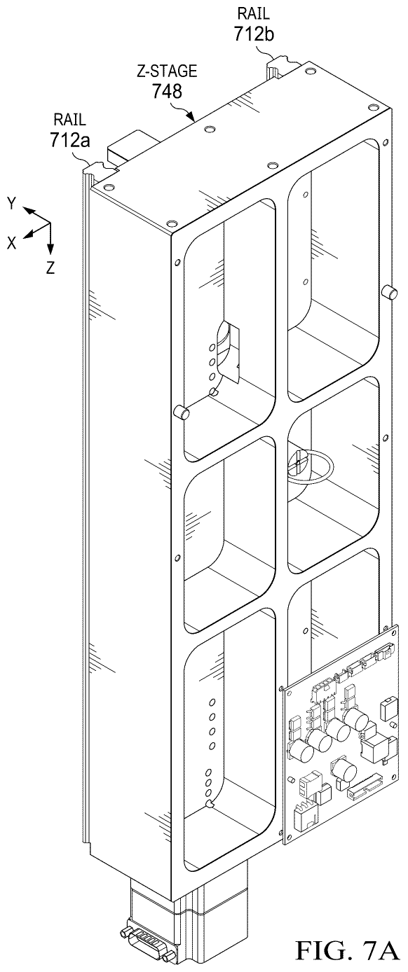

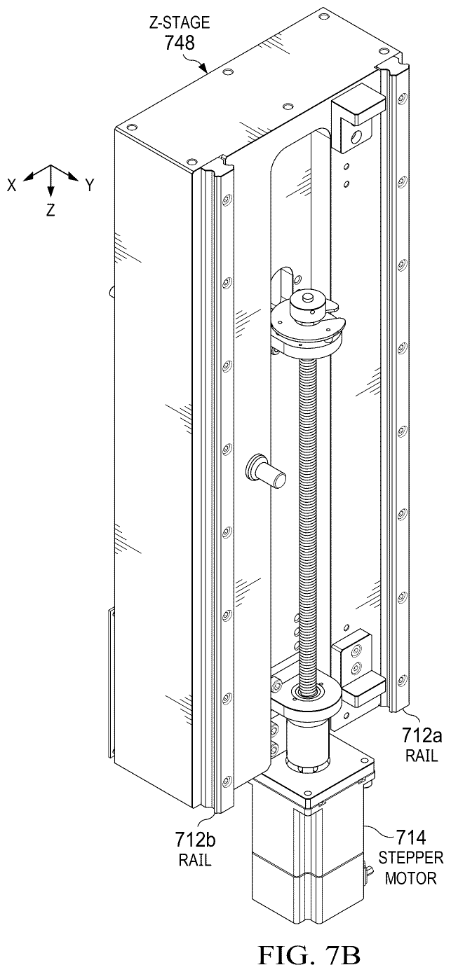

FIGS. 6 to 7B illustrate an example of a z-subsystem, according to at least one embodiment described in the present disclosure. FIG. 6 illustrates an example of a z-stage base 646. The z-stage base 646 includes grooves 610 (610a to 610d). FIGS. 7A and 7B illustrate different views of an example of a z-stage 748. The z-stage 748 includes rails 712 (712a and 712b).

The z-stage 748 may move relative to the z-stage base 646 in any suitable manner. In the example, the groove 610a and the groove 610b of the z-stage base 646 receive the rail 712a of the z-stage 748. Similarly, the groove 610c and the groove 610d of the z-stage base 646 receive the rail 712b of the z-stage 748. The stepper motor 714 moves the z-stage 748 relative to the z-stage base 646.

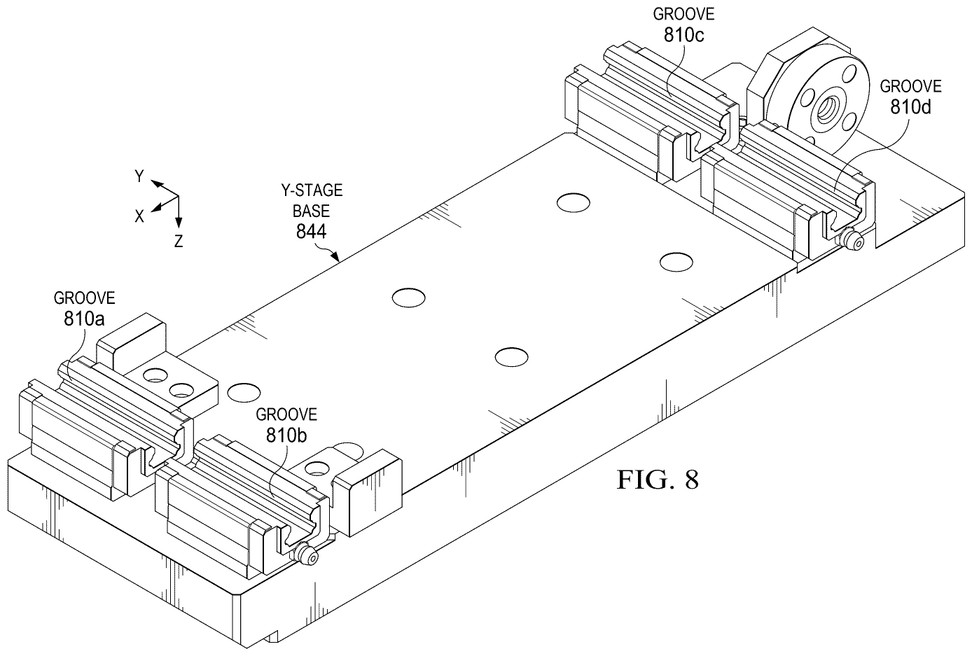

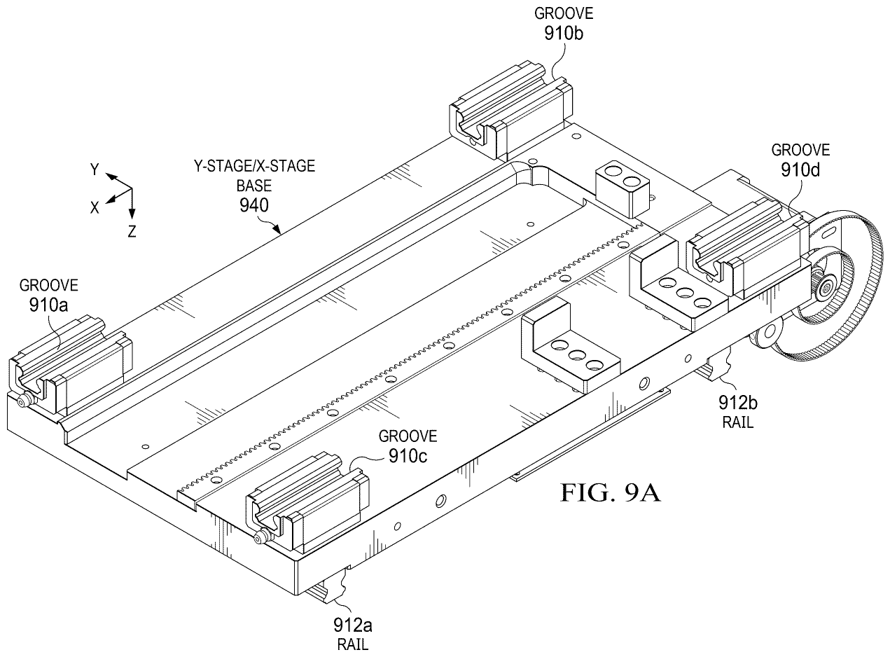

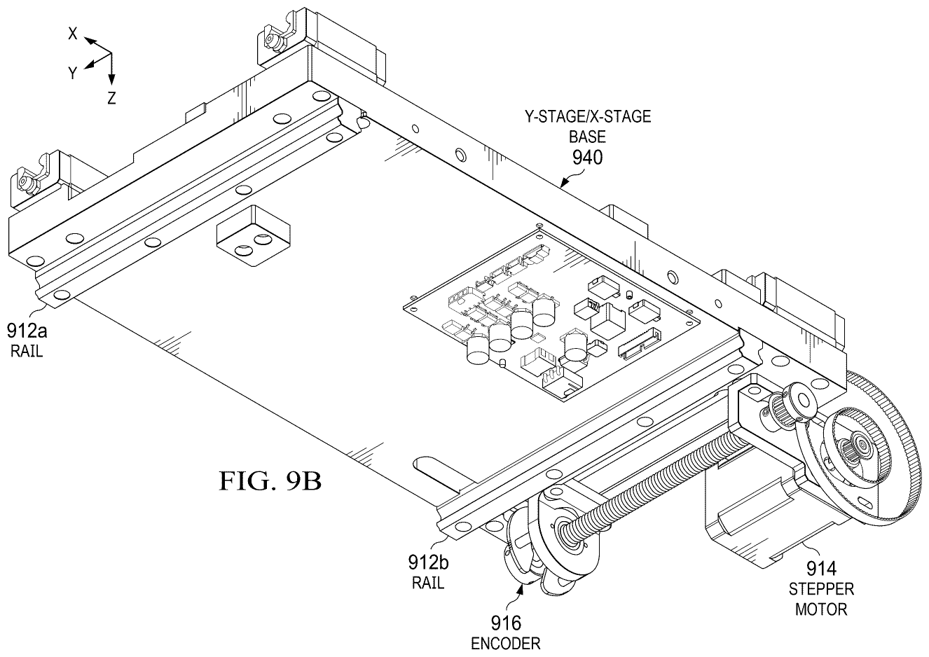

FIGS. 8 to 9B illustrate an example of a y-subsystem, according to at least one embodiment described in the present disclosure. FIG. 8 illustrates an example of a y-stage base 844. The y-stage base 844 includes grooves 810 (810a to 810d). FIGS. 9A and 9B illustrate different views of an example of a y-stage/x-stage base 940. The y-stage/x-stage base 940 includes grooves 910 (910a to 910d), rails 912 (912a and 912b), a stepper motor 914, and a y-direction encoder 916.

The y-stage may move relative to the y-stage base in any suitable manner. In the example, the y-stage/x-stage base 940 (which may operate as a y-stage) moves relative to the y-stage base 844. The groove 810a and the groove 810b of the y-stage base 844 receive the rail 912a of the y-stage/x-stage base 940. Similarly, the groove 810c and the groove 810d of the y-stage base 844 receive the rail 912b of the y-stage/x-stage base 940. The stepper motor 914 moves the y-stage/x-stage base 940 relative to the y-stage base 844.

The y-direction encoder 916 measures movement of the y-stage/x-stage base 940 relative to the y-stage base 844. For example, the y-direction encoder 916 may detect movement of a mechanical device (such as a gear, a step motor, or a lead screw) and translate the movement of the device to linear movement. As another example, the y-direction encoder 916 may detect movement of y-direction magnetic strip of the y-stage/x-stage base 940 and translate the movement of the strip to linear movement. In addition, the grooves 910 (910a to 910d) receive rails of an x-stage, as described in more detail below.

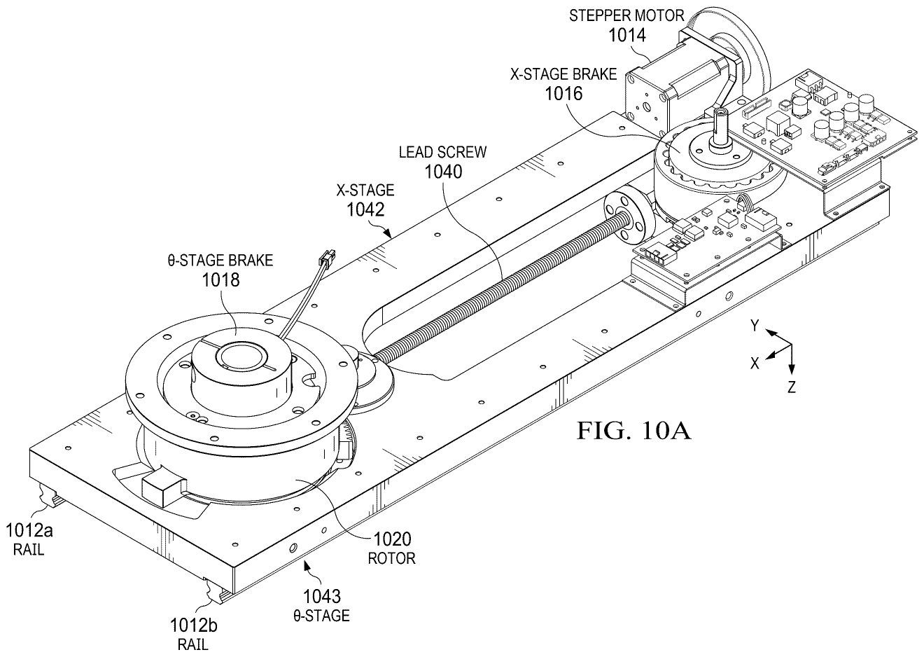

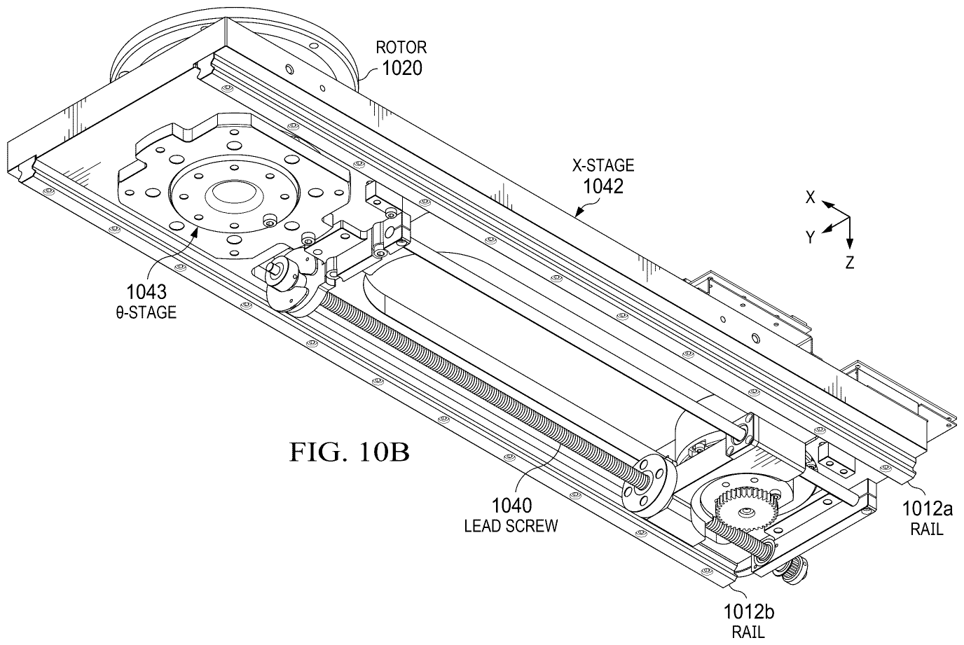

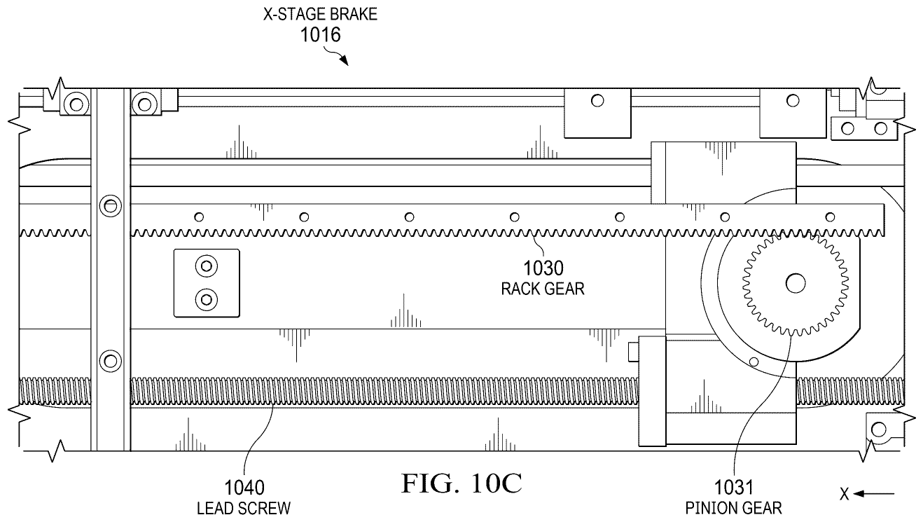

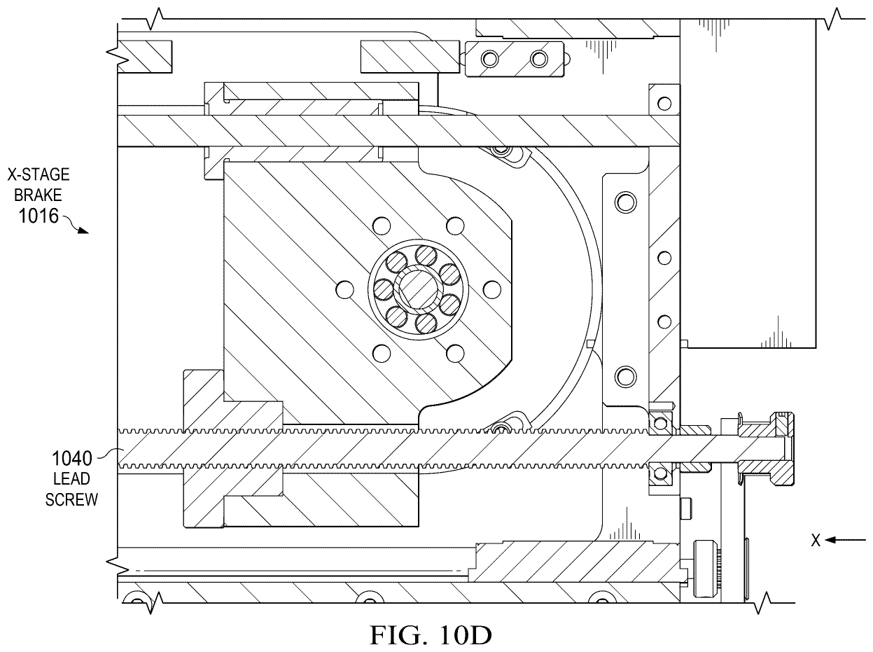

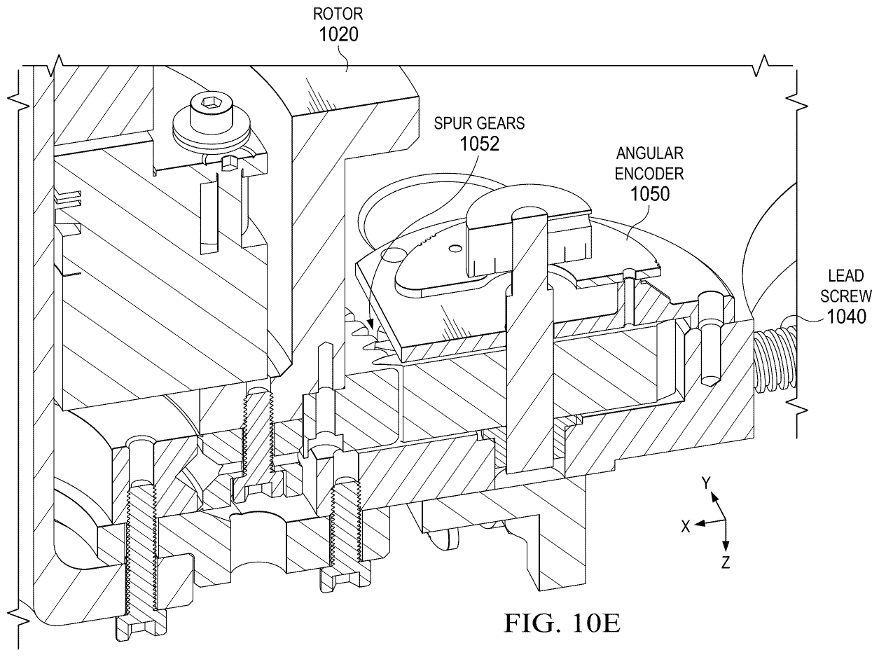

FIGS. 9A to 10E illustrate an example of an x-subsystem and a theta-subsystem, according to at least one embodiment described in the present disclosure. FIGS. 9A and 9B illustrate different views of an example of a y-stage/x-stage base 940, as described above. FIGS. 10A and 10B illustrate different views of an example of an x-stage 1042 and a theta-stage 1043. The x-stage 1042 includes rails 1012 (1012a and 1012b), a stepper motor 1014, and an x-stage brake 1016. The theta-stage 1043 includes a theta-stage brake 1018, a rotor 1020, and a lead screw 1040. FIGS. 10C and 10D illustrate an example of the theta-stage brake 1018 of the theta-stage 1043. The theta-stage brake 1018 includes a rack gear 1030 and a pinion gear 1031. FIG. 10E illustrates an example of an angle encoder 1050 of the x-theta-stage 1043. The angle encoder 1050 includes one or more mechanical gears, e.g., spur gears 1052.

The x-stage may move relative to the x-stage base in any suitable manner. In the example, the x-stage 1042 moves relative to the y-stage/x-stage base 940 (which may operate as an x-stage base). The groove 910a and the groove 910b of the y-stage/x-stage base 940 receive the rail 1012a of the x-stage 1042. Similarly, the groove 910c and the groove 910d of the y-stage/x-stage base 940 receive the rail 912b of the x-stage 1042. The stepper motor 1014 moves the x-stage 1042 relative to the y-stage/x-stage base 940 in the x-direction. The x-stage brake 1016 restricts the movement in the x-direction by, e.g., engaging the rack gear 1030 and the pinion gear 1031. In an example, when a user presses a button on a handle of the delivery arm and the R-stage is fully deployed, the x-stage brake 1016 may open and allow repositioning (e.g., manual repositioning) of the x-stage relative to the y-stage.

The rotor 1020 may move the delivery arm in any suitable manner. In the example, the rotor 1020 rotates relative to a base (such as the theta-stage 1043) to angularly move the delivery arm. The theta-stage brake 1018 restricts angular motion of the delivery arm. In certain embodiments, a computer system may control operation of the theta-stage brake 1018. For example, the computer system may detect a request to allow the angular movement of the delivery arm and then disengage the theta-stage brake 1018 to allow the angular movement. As another example, the computer system may detect a request to restrict the angular movement of the delivery arm and then engage the theta-stage brake 1018 to restrict the angular movement.

The angle encoder 1050 measures movement of mechanical gears, e.g., spur gears 1052, to measure angular motion of the delivery arm. For example, an encoder may include an annular magnet mounted on a rotating shaft that has an array of magnetic sensors statically wrapped around the shaft. The sensors detect and record movement of the shaft relative to the annular magnet.

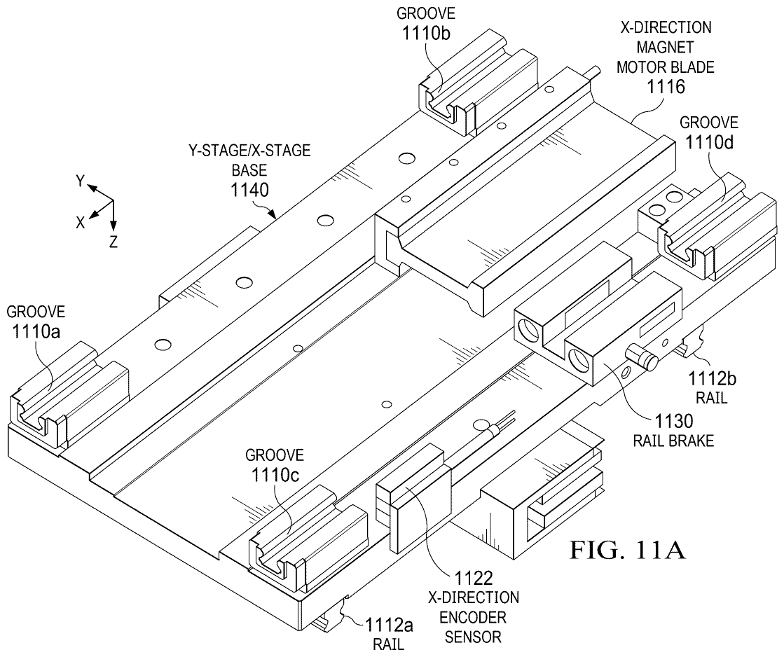

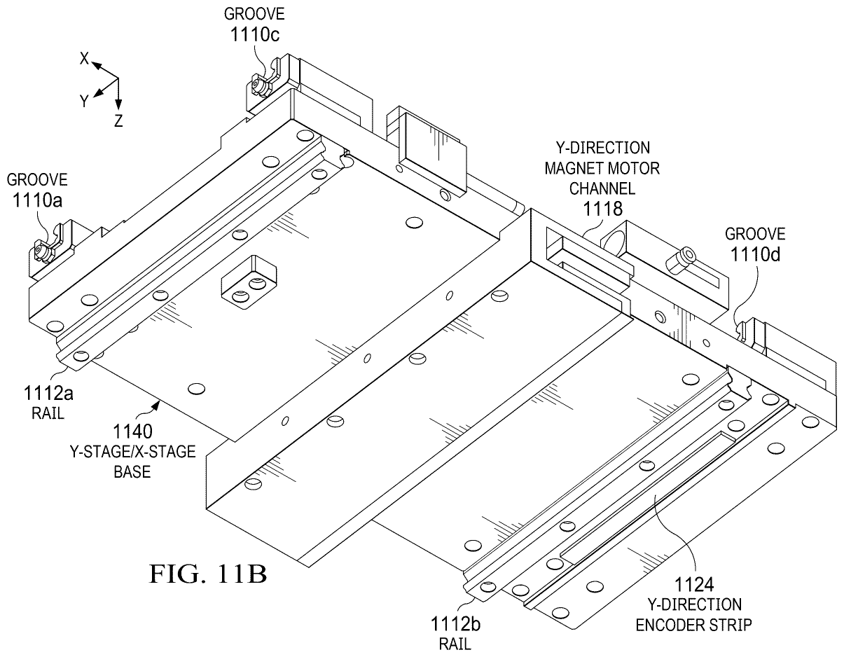

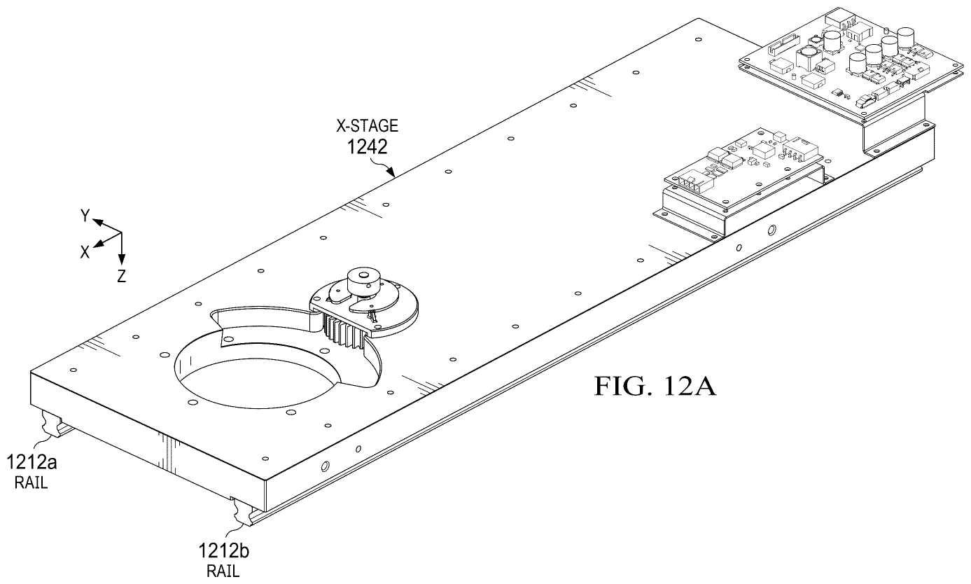

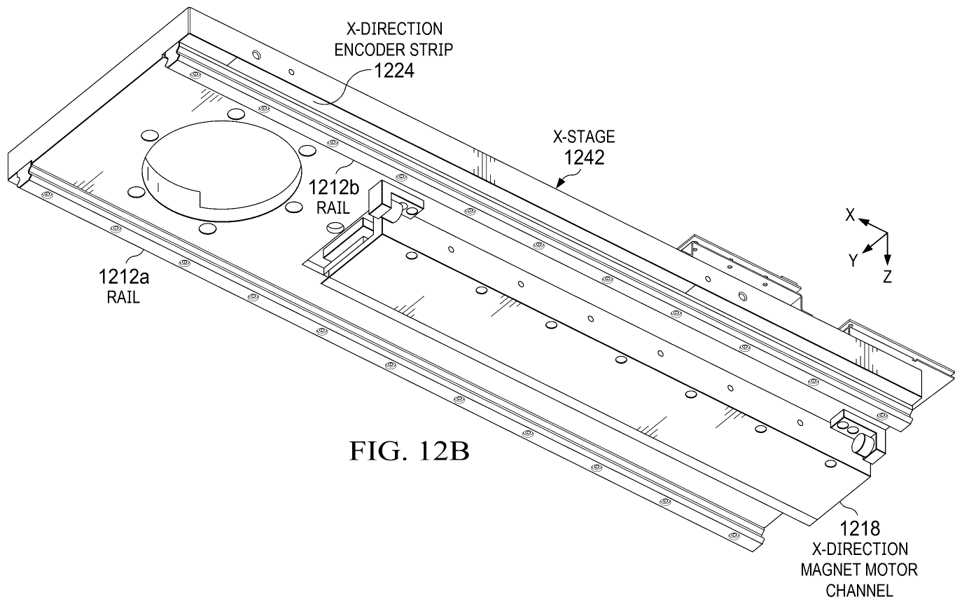

FIGS. 11A to 12D illustrate another example of an x-subsystem, according to at least one embodiment described in the present disclosure. As an example, the subsystems of FIGS. 11A to 12B may be used in place of the subsystems of FIGS. 9A to 10D. FIGS. 11A and 11B illustrate different views of an example of a y-stage/x-stage base 1140. The y-stage/x-stage base 1140 includes grooves 1110 (1110a to 1110d), rails 1112 (1112a and 1112b), an x-direction magnet motor blade 1116, a y-direction magnet motor channel 1118, an x-direction encoder sensor 1122, a y-direction encoder strip 1124, and a rail brake 1130. FIGS. 12A and 12B illustrate different views of an example of an x-stage 1242. The x-stage 1242 includes rails 1212 (1212a and 1212b), an x-direction magnet motor channel 1218, and an x-direction encoder strip 1224. The x-stage 1242 may also include other components, e.g., a stepper motor, an x-stage brake, a theta-stage, a theta-stage brake, a rotor, and/or a rotor base, as described herein.

The x-stage may move relative to the x-stage base in any suitable manner. In the example, the x-stage 1242 moves relative to the y-stage/x-stage base 1140 (which may operate as an x-stage base). The groove 1110a and the groove 1110b of the y-stage/x-stage base 1140 receive the rail 1212a of the x-stage 1242. Similarly, the groove 1110c and the groove 1110d of the y-stage/x-stage base 1140 receive the rail 1112b of the x-stage 1242.

The motion of the x-stage relative to the x-stage base may be generated in any suitable manner. In the example, the x-direction magnet motor channel 1218 receives the x-direction magnet motor blade 1116 of the y-stage/x-stage base 1140. The x-direction magnet motor channel 1218 and the x-direction magnet motor blade 1116 move the x-stage 1242 relative to the y-stage/x-stage base 1140. For example, a variable voltage may be applied to the x-direction magnet motor channel 1218 and/or the x-direction magnet motor blade 1116 to cause the relative motion. The x-direction encoder sensor 1122 detects the linear movement of the x-direction encoder strip 1224 to measure the movement in the x-direction. The rail brake 1130 restricts the movement in the x-direction. For example, the rail brake 1130 may include pads that press against the sides of a rail 1112a and/or 1112b to restrict movement.

In certain embodiments, the y-stage/x-stage base 1140 may operate as a y-stage. For example, the y-direction magnet motor channel 1118 may receive a y-direction magnet motor blade of a y-stage base. As another example, the y-direction encoder strip 1124 may be detected by a y-direction encoder sensor to measure motion in the y-direction.

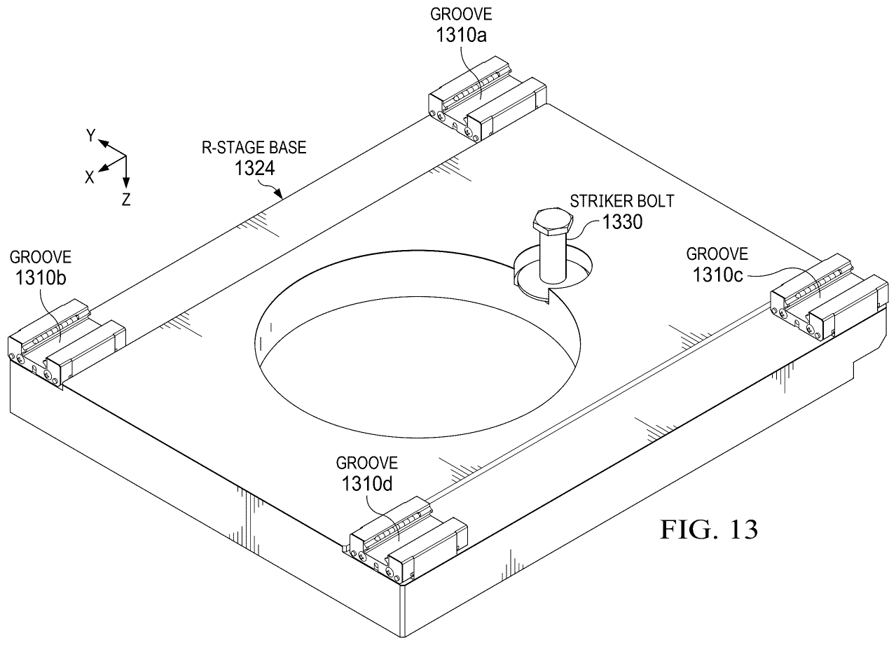

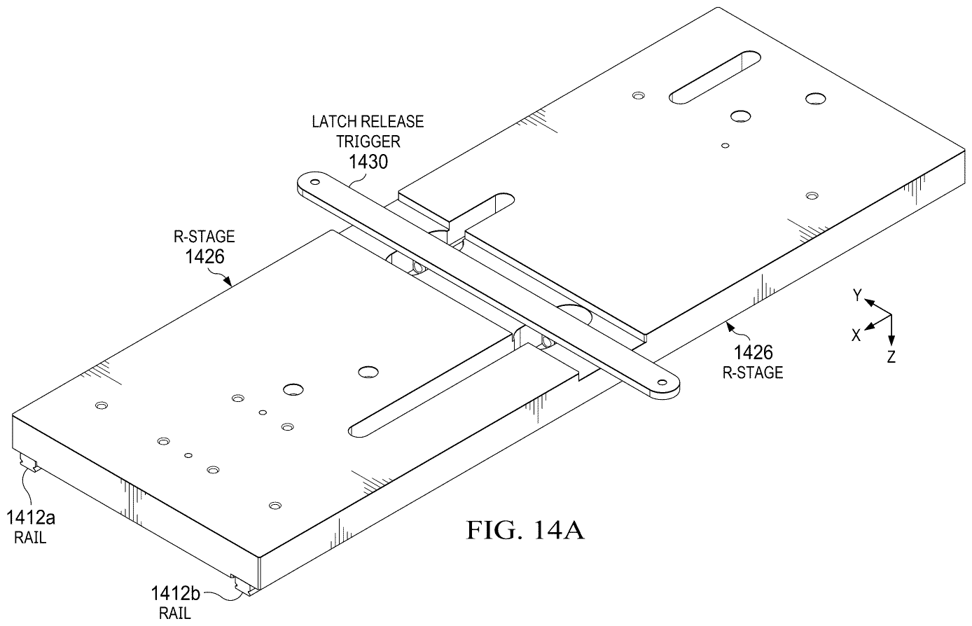

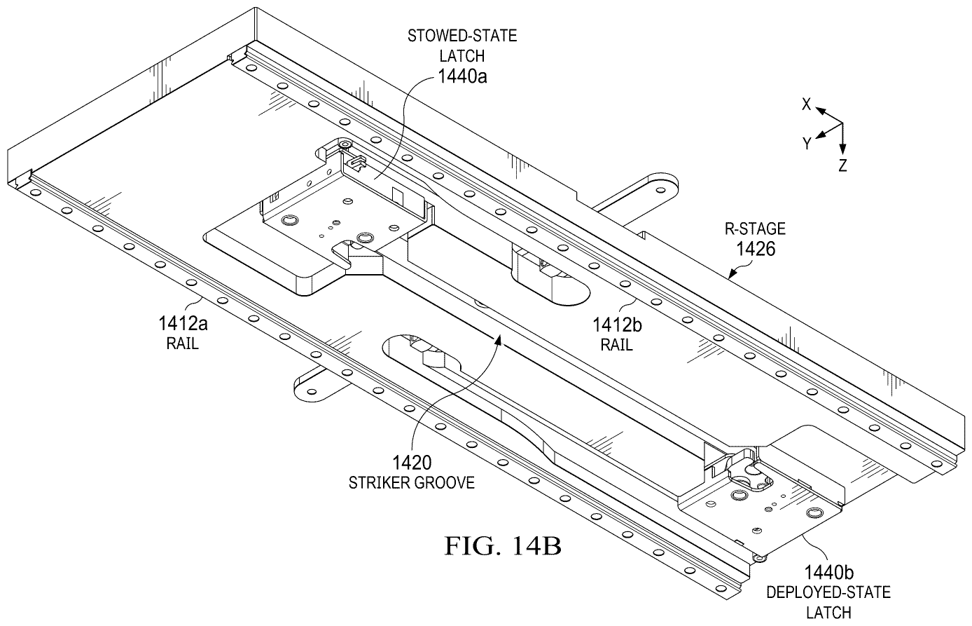

FIGS. 13 to 14B illustrate an example of an R-subsystem, according to at least one embodiment described in the present disclosure. FIG. 13 illustrates an example of an R-stage base 1324. The R-stage base 1324 includes grooves 1310 (1310a to 1310d) and a striker bolt 1330. FIGS. 14A and 14B illustrate different views of an example of an R-stage 1426. The R-stage 1426 includes rails 1412 (1412a and 1412b), a striker groove 1420, a latch release trigger 1430, and latches 1440 (which include a stowed-state latch 1440a and/or a deployed-state latch 1440b).

The R-stage 1426 may move relative to the R-stage base 1324 in any suitable manner. In the example, the groove 1310a and the groove 1310b receive the rail 1412a, and the groove 1310c and the groove 1310d receive the rail 1412b to allow the R-stage 1426 to move relative to the R-stage base 1324. The striker groove 1420 receives the striker bolt 1330.

The motion of the R-stage 1426 relative to the R-stage base 1324 may be allowed and/or restricted in any suitable manner. In certain embodiments, a computer system may control the allowance and/or restriction of the motion. In the example, the latches 1440 (1440a and 1440b) and the striker bolt 1330 may allow and/or restrict movement of the R-stage 1426. When the R-stage 1426 is in a stowed position relative to the R-stage base 1324, the stowed-state latch 1440a receives the striker bolt 1330. The stowed-state latch 1440a may be engaged to restrict movement of the striker bolt 1330, which restricts movement of the R-stage 1426 relative to the R-stage base 1324. The stowed-state latch 1440a may be disengaged to allow movement of the striker bolt 1330, which allows movement of the R-stage 1426 relative to the R-stage base 1324 away from the stowed position. When the R-stage 1426 is in a deployed position relative to the R-stage base 1324, the deployed-state latch 1440b receives the striker bolt 1330. The deployed-state latch 1440b may be engaged to restrict movement of the striker bolt 1330, which restricts movement of the R-stage 1426 relative to the R-stage base 1324. The deployed-state latch 1440b may be disengaged to allow movement of the striker bolt 1330, which allows movement of the R-stage 1426 relative to the R-stage base 1324 away from the deployed position.

In certain embodiments, the latches 1440 may be electromechanical latches that can be controlled by a computer system. For example, the computer system may detect a request to allow movement of the delivery arm from the stowed position towards the deployed position, e.g., a user may press a button on a handle of the delivery arm. In response, the computer system may disengage the stowed-state latch to allow the movement. As another example, the computer system may detect a request to allow movement of the delivery arm from the deployed position towards the stowed position, e.g., the user may again press the button on the handle. In response, the computer system may disengage the deployed-state latch to allow the movement. In some embodiments, the electromechanical latches may be electrically coupled to a button such that the computer system is not used to operate the latches 1440, and depressing the button may engage or release one or both of the stowed state latch 1440a and the deployed state latch 1440b.

While illustrated as having a latch at either end of a channel, it will be appreciated that the latch may slide within the channel such that the R-stage motion may be locked into place in any position along the R-direction motion.

By way of example, a user might grasp a handle, e.g., the handle 332 of FIGS. 3A and/or 3B, and press a button to release the striker bolt 1330 from the stowed-state latch 1440a and release the theta-stage brake 1018. The user may then pull a delivery arm, e.g., the delivery arm 122 of FIG. 1, outwards and at an angle from the chassis. When the user releases the button, the delivery arm 122 may lock into place in the deployed position. The user may then use a joystick, e.g., the joystick 334 of FIGS. 3A and/or 3B, to move the delivery arm 122 in fine-tuned motion to position the laser head over the eye of the patient. For example, the x-stage brake 1016 may be released, and the x-stage 1042 may move a certain coordinated amount while simultaneously a y-stage, e.g., the y-stage 940 of FIGS. 9A and/or 9B, moves a separate coordinated amount to produce the desired motion as indicated by the movement of the joystick.

As another example, for a given surgical setting, a patient may be wheeled towards the left side of an ophthalmic system, e.g., the ophthalmic system 110 of FIG. 1, at a first acute angle. A button may be depressed on a handle, e.g., the handle 332 of FIGS. 3A and/or 3B, located on or near a laser head, e.g., the laser head 240 of FIG. 2, of the system 110, and the laser head 240 may be pulled out using coarse positioning to place the laser head 240 generally over the eye of the patient. A nurse or physician may then use a joystick, e.g., the joystick 334 or 336 of FIGS. 3A and/or 3B, to fine-tune the position of the laser head 240 and/or dock the laser head 240 to the eye of the patient. After the procedure, the laser head 240 may be returned to a stowed position. Within the same surgical setting, another patient may be wheeled directly in front of the ophthalmic system 110, and the laser head 240 may be pulled out in a different direction, but still so as to be over the eye of the patient. In the same surgical setting, another patient may be wheeled towards the right side of the ophthalmic system 110 at a wide angle. The button may be depressed on the handle 332 and the laser head 240 may be pulled out for coarse positioning of the laser head 240 over the eye of the patient. A different joystick 336 or 334, respectively, on the other side of the laser head 340 may be used by the nurse of physician to fine-tune the position of the laser head 240 and dock the laser head 240 to the eye of the patient. After the procedure, the laser head 240 may be returned to the stowed position. In this manner, a surgeon may be provided with a significant amount of flexibility in positioning patients relative to the laser head 240.

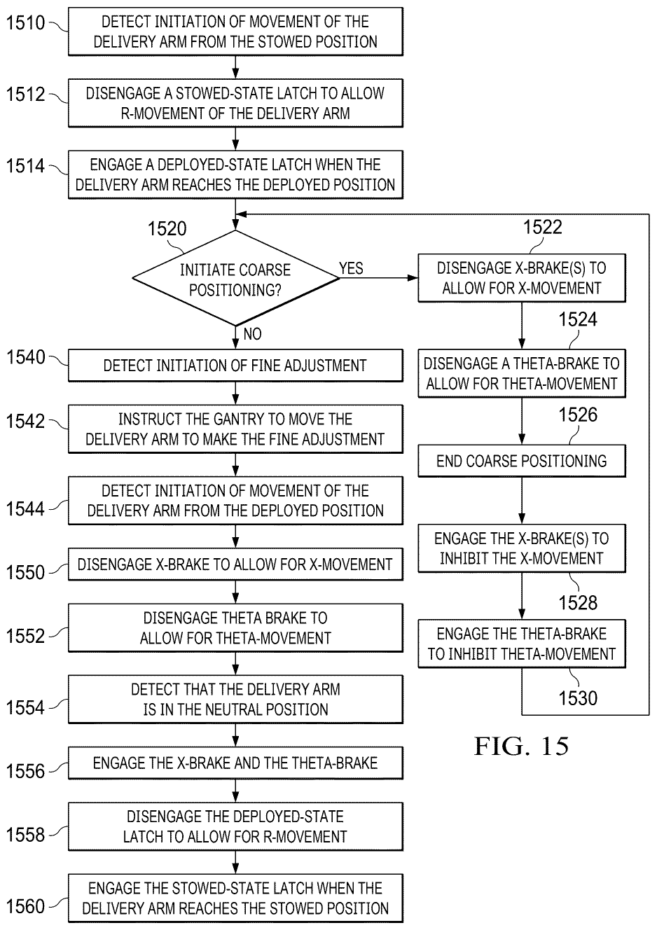

FIG. 15 illustrates an example of a method for moving a delivery arm of an ophthalmic system, according to at least one embodiment described in the present disclosure. In the example, the delivery arm may be coarsely positioned in the general area of the patient's eye and then more finely adjusted to align the laser head of the delivery arm with the eye. The initial coarse positioning and/or subsequent fine adjustment may be performed manually and/or may be automated using an input device. In the example, a user may manually perform the initial coarse positioning by manipulating a handle on the delivery arm. A controller (e.g., a component controller) may automate the subsequent fine adjustment in response to the user moving a joystick.

At block 1510, the controller detects initiation of movement of the delivery arm from a stowed position. For example, the user may select a selector, e.g., press a button on the handle, which activates a switch that is monitored by a controller of a computer system of the ophthalmic system.

At block 1512, the controller disengages a stowed-state latch to allow R-movement of the delivery arm. In the example, disengaging the stowed-state latch allows the user to manually pull the handle in the outward direction towards a deployed position in order to slide an R-stage relative to an R-stage base.

At block 1514, a deployed-state latch is engaged when the delivery arm reaches the deployed position, securing the R-stage relative to the R-stage base. A magnetic sensor of the deployed-state latch may send a signal to the controller indicating the deployed-state latch has engaged.

At block 1520, the user may initiate a coarse positioning process of the delivery arm. For example, the user may continue to press the button after the deployed-state latch engages. If the user initiates the coarse positioning process, the method proceeds to block 1522. If the user does not initiate the coarse positioning process, the method proceeds to block 1540.

At block 1522, the controller disengages one or more x-brake(s) to allow for x-movement of the delivery arm. For example, the controller disengages the x-brake(s) to allow an x-stage to slide relative to an x-stage base.

At block 1524, the controller disengages one or more theta-brake(s) to allow for angular movement of the delivery arm. For example, the controller disengages the theta-brake(s) to allow a theta-stage to rotate relative to a theta-stage base.

At block 1526, the user ends the coarse positioning process. For example, the user releases the button.

At block 1528, the controller engages the x-brake(s) to restrict the x-movement, e.g., to prevent the x-stage from sliding relative to the x-stage base.

At block 1530, the controller engages the theta-brake(s) to restrict the angular movement, e.g., to prevent the theta-stage from rotating relative to the x-stage base. The method may return to block 1520 to check if the user has initiated another coarse positioning process.

At block 1540, the controller detects initiation of fine adjustment of the delivery arm. In certain embodiments, the controller may detect that the user has manipulated the joystick to indicate the movements of the fine adjustment. For example, the user may laterally move the joystick in a particular direction to cause the delivery arm to move in that direction. As another example, the user may rotate the joystick about its axis to move the delivery arm in the z-direction, e.g., counter-clockwise to move the delivery arm upward, clockwise to move the delivery arm downward, or vice-versa.

At block 1542, the controller instructs the gantry to make the fine adjustment of the delivery arm. For example, based on the signals received from the joystick, the controller instructs an x-subsystem, a y-subsystem, and/or a z-subsystem to move the delivery arm.

At block 1544, the controller detects initiation of the movement of the delivery arm from the deployed position to a neutral position. For example, the user may press the button of the handle. The neutral position may be, e.g., the x-stage is at an extreme proximal position and/or the theta-stage is at a neutral angle, such as the angle when the delivery arm is a stowed position.

At block 1550, the controller disengages the x-brake(s) to allow for x-movement of the delivery arm, e.g., to allow the x-stage to slide relative to the x-stage base.

At block 1552, the controller disengages the theta-brake(s) to allow for angular movement of the delivery arm, e.g., to allow the theta-stage to rotate relative to the x-stage base.

At block 1554, the controller detects that the delivery arm is in the neutral position. For example, a sensor may be triggered when the delivery arm is in the neutral position.

At block 1556, the controller engages the x-brake(s) and the theta-brake(s) to prevent x-movement and angular movement, respectively.

At block 1558, the deployed-state latch is disengaged to allow for R-movement. For example, the triggering of the sensor indicating that the delivery arm is in the neutral position may disengage the deployed-state latch to allow the R-stage to move relative to the R-stage base when the delivery arm is moved towards the stowed position.

At block 1560, the stowed-state latch is engaged when the delivery arm reaches the stowed position.

The present disclosure (including the specification, claims, and drawings) includes example embodiments that are intended to aid the reader in understanding the invention and concepts contributed by the inventor to furthering the art and to enable any person skilled in the art to make or use the disclosed embodiments. Modifications (e.g., changes, substitutions, additions, omissions, and/or other modifications) to the embodiments will be readily apparent to those skilled in the art. Accordingly, modifications may be made to the embodiments without departing from the essence of the present disclosure.

In certain instances, modifications may be made to the systems disclosed herein, as apparent to those skilled in the art. For example, parts of a system may be integrated or separated, or an operation of a system may be performed by more, fewer, or other parts. In certain instances, modifications may be made to the methods disclosed herein, as apparent to those skilled in the art. For example, the methods may include more, fewer, or other operations. As another example, certain operations may be optional, combined into fewer operations, or expanded into additional operations. As yet another example, certain operations may be performed in any suitable order or simultaneously.

Furthermore, those skilled in the art will recognize that the present disclosure is not intended to be limited to the example embodiments and that the language of the disclosure is to be accorded the widest scope consistent with the present disclosure. Terms (which may include one or more words) that describe inclusion are generally intended as “open” terms in that they generally do not imply exclusion. For example, the term “including” may be interpreted as “including, but not limited to” or “including at least”; the term “having” may be interpreted as “having, but not limited to” or “having at least”; and the term “comprising” may be interpreted as “comprising, but not limited to” or “comprising at least”, etc.

Additionally, if a specific number is intended, such intent will be explicitly recited in the claim. In the absence of the explicit recitation of a specific number, no such intent is present. If a specific number is explicitly recited, such recitation should be interpreted to mean at least the recited number. For example, the bare recitation of “two Xs”, without other modifiers, may mean “at least two Xs” or “two or more Xs”. Moreover, the use of an indefinite article (e.g., “a” or “an”) or definite article (e.g., “the”) to introduce a noun phrase should not be construed to limit the noun phrase to one, but may be interpreted as an open term “at least one” or “one or more”. This holds even when the same claim includes an open term (e.g., “one or more” or “at least one”) and an indefinite or definite article (e.g., “a” or “an” or “the”).

Moreover, a selection from a list of items should be understood to contemplate a selection of any suitable individual item or any suitable combination of items. For example, the general construction “at least one of A, B, and C” or “one or more of A, B, and C” may include A alone; B alone; C alone; A and B together; A and C together; B and C together; and A, B, and C together. Moreover, any disjunctive term presenting two or more alternative items may be understood to contemplate including one of the items, either of the items, or both items. For example, the general construction “A or B” or “A and/or B” may include A alone, B alone, and A and B together. Additionally, the use of the terms “first,” “second,” “third,” etc. are not necessarily used herein to connote a specific order. For example, the terms “first,” “second,” “third,” etc., may be used to distinguish between different elements.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the Applicants to refer to structures known to those skilled in the art and is not intended to invoke 35 U.S.C. § 112(f).

What is claimed:

1. An ophthalmic system, comprising:
a chassis coupled to a laser source, the laser source configured to generate a laser beam;
a delivery arm coupled to a laser head, the laser head optically coupled to the laser source, the laser head configured to direct the laser beam towards a target;
a gantry coupled to the delivery arm and to the chassis, the gantry comprising:
a positioning system configured to facilitate a motion of the delivery arm, the positioning system comprising an angular system configured to facilitate an angular motion of the delivery arm; and
an adjustment system configured to facilitate the motion of the delivery arm, the adjustment system comprising a plurality of linear subsystems, each linear subsystem of the plurality of linear subsystems configured to facilitate the motion of the delivery arm; and
a computer system configured to:
detect a request for a requested movement of the delivery arm;
determine an angle of an angular movement of the delivery arm relative to the chassis;
generate an instruction for the requested movement according to the request for the requested movement and according to the angle of the angular movement of the delivery arm; and
send the instruction for the requested movement to the adjustment system.

2. The ophthalmic system of claim 1, the computer system configured to generate the instruction for the requested movement according to the request for the requested movement and according to the angle of the angular movement of the delivery arm by:
translating, using the angle, the requested movement expressed in a delivery arm coordinate system to a translated requested movement expressed in a chassis coordinate system; and
generating the instruction for the requested movement using the translated requested movement expressed in the chassis coordinate system.

3. The ophthalmic system of claim 1, wherein:
a chassis coordinate system relative to the chassis comprises a first axis and a second axis, the second axis at a first angle to the first axis;
a delivery arm coordinate system relative to the delivery arm comprises a fourth axis and a fifth axis, the fifth axis at a second angle to the fourth axis; and
the requested movement is expressed in the delivery arm coordinate system, the requested movement comprises a fourth movement relative to the fourth axis and a fifth movement relative to the fifth axis.

4. The ophthalmic system of claim 3, the computer system configured to generate the instruction for the requested movement according to the request for the requested movement and according to the angle of the angular movement of the delivery arm by:
calculating a first movement relative to the first axis according to the fourth movement, the fifth movement, and the angle of the angular movement of the delivery arm;
calculating a second movement relative to the second axis according to the fourth movement, the fifth movement, and the angle of the angular movement of the delivery arm; and
generating the instruction for the requested movement according to the first movement and the second movement.

5. The ophthalmic system of claim 1, the plurality of linear subsystems of the adjustment system comprising:
a first subsystem configured to move the delivery arm in a first direction relative to a first axis of a chassis coordinate system relative to the chassis; and
a second subsystem configured to move the delivery arm in a second direction relative to a second axis of the chassis coordinate system.

6. The ophthalmic system of claim 1, the plurality of linear subsystems of the adjustment system comprising:
a third subsystem configured to move the delivery arm in a third direction parallel to a third axis, the third axis defined according to an optical axis of the laser head of the delivery arm.

7. The ophthalmic system of claim 1, a linear subsystem of the plurality of linear subsystems comprising:
a stage base; and
a stage configured to move relative to the stage base to facilitate the motion of the delivery arm.

8. The ophthalmic system of claim 7, the linear subsystem of the plurality of linear subsystems comprising:
an encoder configured to measure a linear movement of the stage relative to the stage base.

9. The ophthalmic system of claim 1, the angular system of the positioning system comprising:
a rotary stage; and
a rotor configured to rotate relative to the rotary stage to enable the angular motion of the delivery arm.

10. The ophthalmic system of claim 1, the positioning system comprising:
an encoder configured to measure the angle of the angular movement of the delivery arm.

11. The ophthalmic system of claim 1, wherein the angular system is coupled to a linear subsystem of the plurality of linear subsystems.

12. The ophthalmic system of claim 1, the positioning system comprising:
a fourth subsystem configured to enable a fourth linear motion of the delivery arm in a fourth direction relative to a rotational axis of the delivery arm.

13. The ophthalmic system of claim 1, the positioning system comprising:
an angular brake configured to restrict the angular motion of the delivery arm.

14. The ophthalmic system of claim 1, the computer system configured to:
detect a request to allow a motion of the delivery arm between a stowed position and a deployed position; and
disengage a stowed-state latch to allow the motion of the delivery arm from the stowed position towards the deployed position.

15. The ophthalmic system of claim 1, comprising:
a latch configured to restrict a motion of the delivery arm from a stowed position.

16. The ophthalmic system of claim 1, comprising:
a latch configured to restrict a motion of the delivery arm from a deployed position.

17. The ophthalmic system of claim 1, the computer system configured to:
detect a request to allow the angular motion of the delivery arm; and
disengage one or more brakes to allow the angular motion of the delivery arm.

18. A method comprising:
positioning a first patient for a first laser-based eye procedure at a first orientation relative to a laser surgical device in a surgical setting;
providing first input to a first joystick of the laser surgical device to change a position of a delivery head of the laser surgical device to a first position, the first input causing a first corresponding and coordinated motion of two distinct linear motion actuators operating in two orthogonal directions;
after the laser surgical device is positioned in the first position, performing the first laser-based eye procedure;
without repositioning a chassis of the laser surgical device, positioning a second patient for a second laser-based eye procedure at a second orientation relative to the laser surgical device in the surgical setting, the second orientation different from the first orientation;
providing second input to a second joystick of the laser surgical device to change the position of the delivery head of the laser surgical device to a second position, the second input causing a second corresponding and coordinated motion of the two distinct linear motion actuators operating in the two orthogonal directions; and
after the laser surgical device is positioned in the second position, performing the second laser-based eye procedure.
